(19) United States
(12) Patent Application Publication
(10) Pub. No.: US 2009/0163831 A1
(43) Pub. Date: Jun. 25, 2009

(54) Methods for the detection of ultraviolet light reactive alternative cellular energy pigments (ACE-pigments)

(75) Inventor: William John Martin, South Pasadena, CA (US)

Correspondence Address: William John MARTIN, 1634 Spruce Street, South Pasadena, CA, 91030, US

(73) Assignee: MI Hope Inc. dba Progressive University, South Pasadena, CA (US)

(21) Appl. No.: 12/005,185

(22) Filed: Dec. 24, 2007

(51) Int. Cl. A61B 5/00; G01N 21/64; G01N 21/75

(52) U.S. Cl. 600/573; 436/172; 436/164

(57) ABSTRACT

Ultraviolet light reactive alternative cellular energy pigments (ACE-pigments) are produced in response to certain types of viral infections, and in other pathological circumstances. ACE-pigments have energy transducing activities, and in particular have the capacity to convert various forms of physical energies, such as electromagnetic radiations, magnetic force and sound frequencies, into chemical energy (both reducing and oxidizing). They are thought to play a metabolic supportive role in the virus infected cells. ACE-pigments can be eliminated from the body through the skin and can also be found in and attached to hair. A method is described for collecting ACE-pigments from the skin and bodily fluids of a patient and viewing the ACE pigments using ultraviolet light in the presence of a dye such as neutral red, which enhances their ultraviolet (UV) induced fluorescence. The described fluorescence examination provides a means of easily assessing the ACE pathway in patients and can provide a means of monitoring the usefulness of administering various means to further energize this pathway.

### CROSS REFERENCE TO PREVIOUSLY SUBMITTED BUT NOW ABANDONED PATENT APPLICATIONS

Ser. No. 10/044,683. Therapy of stealth virus associated cancers and other conditions using light. William John Martin. (Abandoned)

Ser. No. 10/047,313. Therapy of stealth virus associated cancers and other conditions using medium chain triglycerides. William John Martin. (Abandoned)

Ser. No. 10/050,232. Diagnosing and monitoring the therapy of stealth virus infections based on the detection of auto-fluorescent material in hair. William John Martin. (Abandoned)

Ser. No. 10/058,480. Therapy of stealth virus associated cancers and other conditions using magnetic energy. William John Martin. (Abandoned)

Ser. No. 10/164,258 Energy supportive therapy of stealth virus associated diseases. William John Martin. (Abandoned)

Ser. No. 10/174,466 Sound therapy of stealth virus associated diseases. William John Martin. (Abandoned)

Ser. No. 10/192,936 ACE-Pigments and humic acids as energy sources. William John Martin. (Abandoned)

Submitted: Ser. No. 10/______ Methods for Collection of Alternative Cellular Energy Pigments (ACE-pigments). William John Martin. (Abandoned)

Submitted: Ser. No. 10/______ Methods for Elimination of Toxic Alternative Cellular Energy Pigments (ACE-pigments) and for Their Replacement Using Activated Humates, Including Humic and Fulvic Acids. William John Martin. (Abandoned)

### UNITED STATES PATENTS (AWARDED)

U.S. Pat. No. 5,985,546 Stealth virus detection in the chronic fatigue syndrome. William John Martin

U.S. Pat. No. 8,891,468 Stealth virus detection in the chronic fatigue syndrome. William John Martin

U.S. Pat. No. 5,752,488 Isolated stealth viruses and related vaccines. William John Martin

U.S. Pat. No. 5,703,221 Stealth virus nucleic acids and related methods. William John Martin

U.S. Pat. No. 6,368,637. Method and composition for topical treatment of viral lesions. Jon Stoneburner

### PCT (PATENT COOPERATION TREATY)

WO 92/20797 Stealth virus detection in the chronic fatigue syndrome. William John

WO 99/34019 Stealth virus nucleic acids and related methods. William John Martin

WO 99/60101 Stealth viruses and related vaccines. William John Martin

### REFERENCES TO PUBLISHED ARTICLES

### Alternative Cellular Energy Pigments (ACE-Pigments)

1 Martin W J. Alternative cellular energy pigments mistaken for parasitic skin infestations. Exp. Mol. Path 78: 212-214, 2005.
2 Martin W J. Alternative cellular energy pigments from bacteria of stealth virus infected individuals. Exp. Mol. Path 78: 217-217, 2005.
3 Martin W J. Progressive Medicine. Exp Mol Path 78: 218-220, 2005.
4 Martin W J, Stoneburner J. Symptomatic relief of herpetic skin lesions utilizing an energy based approach to healing. Exp. Mol. Path 78: 131-4, 2005.
5 Martin W J. Etheric Biology. Exp Mol Path 78: 221-227, 2005.
6 Martin W J. Stealth Virus Culture Pigments: A Potential Source of Cellular Energy. Exp. Mol. Pathol. 74: 210-223, 2003.
7 Martin W J. Complex intracellular inclusions in the brain of a child with a stealth virus encephalopathy. Exp. Mol. Pathol. 74: 179-209, 2003.
8 Martin W J. Photons and phonons: Theoretical aspects of biophysics and potential therapeutic applications. Proceeding of Neural Therapy Workshop on Sound and Light Therapy, Seattle, Wash., Feb. 21-23, 2003.

### Stealth Adapted Viruses

1 Martin W J Chronic fatigue syndrome among physicians. A potential result of occupational exposure to stealth viruses. Explore 2001; 10: 7-10.
2 Martin W J. Stealth Viruses. Explore 2001; 10: 17-19.
3 Durie G M, Collins R. Martin W J. Positive stealth virus cultures in multiple myeloma. A possible explanation for neuropsychiatric co-morbidity. Presented at the Am. Soc. Hematology annual meeting October 2000.
4 Martin W J. Chemokine receptor-related genetic sequences in an African green monkey simian cytomegalovirus-derived stealth virus. Exp Mol Pathol. 2000; 69:10-6.
5 Martin W J., Anderson D. Stealth virus epidemic in the Mohave Valley: severe vacuolating encephalopathy in a child presenting with a behavioral disorder. Exp Mol Pathol. 1999; 66:19-30.
6 Martin W J. Melanoma growth stimulatory activity (MGSA/GRO-alpha) chemokine genes incorporated into an African green monkey simian cytomegalovirus-derived stealth virus. Exp Mol Pathol. 1999; 66:15-8.
7 Martin W J. Bacteria-related sequences in a simian cytomegalovirus-derived stealth virus culture. Exp Mol Pathol. 1999; 66:8-14.
8 Martin W J. Stealth adaptation of an African green monkey simian cytomegalovirus. Exp Mol Pathol. 1999; 66:3-7.
9 Martin W J. Cellular sequences in stealth viruses. Pathobiology 1998; 66:53-8.
10 Martin W J. Detection of RNA sequences in cultures of a stealth virus isolated from the cerebrospinal fluid of a health care worker with chronic fatigue syndrome. Case report. Pathobiology. 1997; 65:57-60.
11 Martin W J., Anderson D. Stealth virus epidemic in the Mohave Valley. I. Initial report of virus isolation. Pathobiology. 1997; 65:51-6.
12 Martin W J. Simian cytomegalovirus-related stealth virus isolated from the cerebrospinal fluid of a patient with bipolar psychosis and acute encephalopathy. Pathobiology. 1996; 64:64-6.
13 Martin W J. Stealth viral encephalopathy: report of a fatal case complicated by cerebral vasculitis. Pathobiology. 1996; 64:59-63.
14 Martin W J. Genetic instability and fragmentation of a stealth viral genome. Pathobiology. 1996; 64:9-17.
15 Martin W J. Severe stealth virus encephalopathy following chronic-fatigue-syndrome-like illness: clinical and histopathological features. Pathobiology. 1996; 64:1-8.
16 Martin W J. Stealth virus isolated from an autistic child. J Autism Dev Disord. 1995; 25:223-4.
17 Gollard R P, Mayr A., Rice D A, Martin W J. Herpesvirus-related sequences in salivary gland tumors. J Exp Clin Cancer Res., 1996; 15: 1-4.
18 Martin W J., Glass R T. Acute encephalopathy induced in cats with a stealth virus isolated from a patient with chronic fatigue syndrome. Pathobiology. 1995; 63:115-8.
19 Martin W J, et al. African green monkey origin of the atypical cytopathic ‘stealth virus’ isolated from a patient with chronic fatigue syndrome. Clin Diag Virol 1995: 4: 93-103.
20 Martin W J. Stealth viruses as neuropathogens. CAP Today. 1994; 8: 67-70.
21 Martin W J. et al. Cytomegalovirus-related sequence in an atypical cytopathic virus repeatedly isolated from a patient with chronic fatigue syndrome. Am J. Pathol. 1994; 145: 440-51.

### STATEMENT REGARDING FEDERALLY SPONSORED RESEARCH OR DEVELOPMENT

No Federal funding was received in support of the research covered in this patent application.

### REFERENCE TO SEQUENCE LISTING, A TABLE, OR A COMPUTER PROGRAM LISTING COMPACT DISK APPENDIX

None provided.

### BACKGROUND OF THE INVENTION

With the exception of chlorophyll-containing plants and bacteria, it has been widely believed that virtually all of the energy produced by living structures is derived from the catabolic (metabolic breakdown) of food. The applicant has proposed that an additional source of cellular energy is available to living cells through the conversion of physical energies, including electromagnetic radiation, magnetic fields, sound, etc., to biological energy, including the capacity to perform biochemical reactions. This energy conversion (transduction) is mediated by mineral-containing, pigmented materials that the applicant has termed “alternative cellular energy pigments” (ACE-pigments). These pigments were identified as intracellular inclusions developing in cell cultures infected with cell damaging (cytopathic) viruses that can be isolated from patients with a range of neuropsychiatric and other illnesses. The viruses are termed “stealth” or “stealth-adapted” because they do not evoke an inflammatory reaction within the infected individuals. They are molecularly heterogeneous, although some are clearly derived from herpes viruses, including African green monkey simian cytomegalovirus (SCMV). The pigmented structures seen were conglomerates of smaller microscopic particles. They could also take the form of long threads and ribbon-like structures. ACE-pigments showed various properties indicative of broadly based energy transducing activities. Specifically, they were usually autofluorescent, electrostatic and occasionally ferromagnetic. Smaller particles would commonly display movements within a fluid environment that far exceeded that explainable as Brownian movement. Larger particles would occasionally display slow oscillatory movements and could usually be induced to vibrate when exposed to a particular sound frequency that differed between different particles. ACE-pigments could also “reduce” (donate electrons to) specific acceptor molecules such as the tetrazolium salt MTT, while at the same time “oxidize” (remove electrons from) other molecules such as in the conversion of ascorbic acid to oxalic acid. Ultraviolet (UV) light induced fluorescence was enhanced in the presence of certain dyes and the particles could differentially alter the emitted fluorescence of known UV fluorescent dyes such as acridine orange. Stealth virus infected cells stained with acridine orange contained finely dispersed material that displayed multi-colored (green-yellow and red) fluorescence, not seen in uninfected cells. The fluorescing materials were also present in culture supernatants. Their movement properties could be readily followed using acridine orange staining. Enhanced fluorescence of cells and extra-cellular materials was also seen using the supravital stain neutral red, which itself shows no UV induced fluorescence.

Of particular note was that the formation of ACE-pigments clearly correlated with a lessening of the cytopathic effect (CPE) caused by the stealth viruses. Thus, for example, removal of the ACE-pigments from the cultures resulted in a reactivation of the CPE that could be prevented by re-addition of isolated ACE pigments to the re-feeding culture medium.

Gas bubbles, presumably originating from electrolysis, could occasionally be seen coming from larger ACE-pigment particles placed within a water droplet. The particles can bind with both anion exchange and cation exchange resins. These various properties were strongly influenced by prior exposure to the various energy sources listed above. Individual particles also behaved differently over time, including the gain or loss of motility, ferromagnetism and both auto and stain enhanced fluorescence. The impression was gained of that of miniature batteries that could exist in an energy reactive (chargeable) form or as non-reactive forms. It was possible that the non-reactive state reflected a situation in which the ACE-pigments were fully charged and, therefore, unable to respond to further energy input.

Similar physical and energy transducing properties were identified with the naturally occurring compounds humates, humic and fulvic acids. Recent studies on these compounds show they consist of a lattice of minerals coordinating (bonding) mainly with aromatic molecules including pyrroles, porphyrins, amino acids (tyrosine, tryptophan, phenylalanine and histidine), nucleotides and carbohydrates. The minerals include both metals and non-metals. The composition of humates generally reflects the relative availability of the various minerals and aromatic and other compounds. Similar to ACE-pigments obtained from stealth virus cultures, humic/fulvic acids display the capacity is to act as semi-conductors. Specifically, they can allow for the intramolecular movement of electric charge such that electron rich negatively charged (n) regions can, under the influence of externally applied energies, become separated from electron poor positive charged (p) holes. This charge separation imparts the electrostatic properties on these substances. Individual particles can also show considerable movements well beyond that explainable as Brownian movement.

ACE-pigments were similarly shown by energy dispersive analysis (EDX) to contain a wide array of various minerals, often in limited forms within individual particles. The culture supernatants also contained a wide diversity of unusual chemical compounds on gas chromatographic—mass spectroscopy (GC-MS). Many of the compounds were not fully identifiable on even the largest database of GC-MS identifiable chemicals available at California Institute of Technology (CalTech) in Pasadena, Calif. Generally, however, most of the compounds were aromatic with potential sites for coordination with metals.

ACE-pigments have been seen in the hair and nails of stealth virus infected patients. They were initially identified on the basis of ultraviolet light induced autofluorescence. Larger particles clearly comprised a conglomerate of finer material that would show as discrete fluorescent areas within the overall particles. ACE pigments can also be seen as minute skin and hair deposits in certain stealth virus infected patients. Most likely, microscopic particles pass onto the skin as a component of normal perspiration. Because of electrostatic and possibly other forms of selective mineral affinity forces, the microscopic particles can coalesce into various conglomerates that can be seen as visible particles. Several patients have reported finding such particles on their bed sheets following a night's sleep. Over the past 15 years, the applicant has been aware of several patients inadvertently being told they have head lice, for which “Lindane” containing shampoo was prescribed. This compound can be especially neurotoxic for patients with an already damaged brain due to the stealth virus infection. Psychiatric patients also not uncommonly report what they perceive as skin infestation, and are labeled as having a delusional parasitosis. There is little doubt that the elimination of ACE-pigments through the skin has essentially gone unnoticed by the medical profession.

It is unlikely that ACE pigments are restricted to patients infected with stealth adapted viruses. It was interesting, therefore, to learn from Dr. Jon Stoneburner, OD, of UV light induced fluorescent materials in the vicinity of skin lesions caused by Herpes simplex virus (HSV), Herpes zoster virus (HZV) and human papillomavirus (HPV). The skin lesions had to be first painted with neutral red dye. The fluorescence was considered as coming from the actual viruses and merely an incidental observation to the ability of the dye/light combination to expedite the healing of the viral lesions. The UV light induced fluorescence markedly fades over 15-30 minutes and can not be further evoked by the further addition of neutral red. This was attributed by Dr. Stoneburner to the destruction of the viruses. Dr. Stoneburner continues to use tube fluorescent light sources without filtering the light. Based on my suggestions and direction, Dr. Stoneburner confirmed that fluorescent material can be collected onto Q-tips applied directly to herpesvirus lesions. Again, under my direction, he has provided confirmation that what he now appreciates as ACE pigments are produced throughout the body and that the pigments apparently transform from a fluorescent to a non-fluorescent form.

I hypothesize that ACE-pigments existed in at least three states: i). Directly fluorescent when exposed to UV light; ii) responsive to UV light by fluorescence only in the presence of an enhancing dye such as neutral red; iii) non-responsive to either direct or dye enhanced UV illumination either because of being non-chargeable or being more fully charged.

Further studies on the functions and compositions of ACE-pigments in individual patients would be facilitated if there were reliable means of collection of such particles. Using isolated ACE-pigments, it will be possible to define their energy absorbing and transducing properties and to more clearly distinguish between reactive (energy absorbing) forms from non-reactive and potentially more fully charged forms. Moreover, in individual patients it would be of interest to assess the status of the ACE pathway by determining if energy chargeable forms existed that could be potentially energized using UV light or other forms of electromagnetic radiations ranging from gamma waves to radio waves. Similarly, the magnetic and sound energy responsiveness can be determined. Composition can be determined using various spectroscopic and elemental analyses. The present invention describes several methods for isolating and assessing UV responsiveness of ACE-pigments obtained from the skin, blood, saliva and urine of individuals.

(References to published articles on Alternative Cellular Energy Pigments (ACE-pigments), Stealth Adapted Viruses and Use of Neutral Red in therapy of herpesvirus infections are listed above. Also included are a series of now abandoned patent applications that provide additional documentation of the findings leading to this present application. Information in all of the published references and cited awarded and abandoned patents applications of William John Martin are incorporated into the present application.

### BRIEF SUMMARY OF THE INVENTION

The invention discloses a means of assessing the status of the alternative cellular energy (ACE) pathway in patients. More specifically, it describes methods for the collection of materials, termed ACE pigments, from skin, urine and saliva and determining whether such materials are reactive to ultraviolet (UV) light, either when directly illuminated with UV light or illuminated in the presence of a suitable dye such as neutral red. The presence of UV reactive ACE pigments can indicate the need for therapeutic interventions aimed at enhancing the ACE pathway in order to restore non-reactivity to UV light of the body's ACE pathway.

The presence of reactive ACE pigments is defined for the purpose of this application as materials generated by the patient that exhibits fluorescence when either directly exposed to UV light or when exposed to UV light after contact a suitable dye such as neutral red. The preferred means of illumination is a compact spiral fluorescent light that emits UV-A light such as is provided by the ProLume or other comparable light. The detection of fluorescence is enhanced by viewing the UV illuminated material in the absence of visible light. This can be achieved using a UV transmission filter that blocks all of the visible light emitted by the UV light source. A suitable dye is neutral red, which does not fluoresce by itself but does promote the fluorescence of reactive ACE pigments. The preferred approach is to stain a Q-tip or other material used for collecting ACE pigments with a 1 mg/ml solution of neutral red and to look for UV induced fluorescence. Neutral red can also be directly added to saliva or urine specimens followed by UV illumination.

### BRIEF DESCRIPTION OF THE DRAWINGS

None included

### DETAILED DESCRIPTION OF THE INVENTION

The present invention describes improved methods to enhance the detection of reactive alternative cellular energy pigments (ACE-pigments) from a patient suspected of having such pigments. The improvements consist of 1) Using aluminum cone shielded compact spiral ultraviolet light bulb. 2) An UV light transmission filter that blocks visible light and allows for a very dark background to view any UV induced fluorescence. 3) Using neutral red, or another suitable dye that can markedly increase the fluorescence of reactive ACE pigments. Using these improvements, UV reactive ACE pigments can be more readily detected in patients and can provide a basis for assessing the potential therapeutic usefulness of further activating the ACE pathway in patients.

The preferred compact spiral bulb is that being marketed under the name ProLume by Halco Lighting (catalogue number PL15SE/BLB). It is listed by Underwriters Laboratory (UL) as 88DN, E217444 and is 15 Watts. Its UV emission spectrum is that of a typical mercury tube BLB light and extends from 345 nm to 400 nm with a well defined peak at 366 nm. It also emits three minor peaks within the blue visible light range at 405, 408 and 437 nm. Consequently, the bulb shines blue. The major advantage of the fluorescent spiral bulb over a tube light of comparable power is that it provides a more compact source of light that can be further concentrated by using aluminum or other UV-A reflective material fashioned into a cone shape.

The detection of fluorescence is best observed against a black background. The above light shines blue because of the emission spectrum extending above 400 nm. This component can be effectively blocked using a UV transmission filter. Suitable examples are Kodak Wratten 18A filter, Schneider Optics, New York, filter code number 403; and Rolyn Optics Company, Covina Calif., catalogue number 65.1030. The later filter comes in 50 mm×50 mm square glass plate. It blocks all light transmission from 400 nm to 700 nm. The filter is placed in the path of the UV-A light being emitted from the ProLume compact UV-A fluorescent bulb so that only the UV-A light component is transmitted. The filter was held in place by the aluminum shielding used to focus the UV-A light to an outlet that is covered by the filter.

The UV fluorescent properties of ACE pigments can be enhanced by brief exposure to a solution containing neutral red dye that is not oxidized as shown by the formation of fine crystals. Freshly repaired dye can conveniently be prepared by using a Q-tip to collect approximately 5 mg of dye and stirring the Q-tip into 5 ml of tap water. Neutral red does not fluoresce by itself but can strongly promote the fluorescence of ACE pigments. The actual concentration of neutral red is not critical but should not overpower any observed fluorescence.

In summary, a Q-tip or other material is used for collecting ACE pigments from the patient's skin. It is then stained with a neutral red solution and observed in the dark for fluorescence evoked by a shielded ProLume UV emitting light that is passed through a UV transmission but visible light blocking filter. The fluorescence can range from yellow to bright pink or red. Control material that has not been used to collect ACE pigments can be used as a negative control.

Various modifications and adaptations of this basic approach will be apparent to any practitioner. These include the use of a wide range of collection materials; other dyes; other UV light sources, including fluorescent microscopes. Neutral red can also be placed on the skin or even small amounts injected into the skin and the area illuminated using a UV light source

The hot bath method can be used as a means of promoting the excretion of ACE-pigments as can any other means of inducing excess perspiration (such as heat and exercise). In one such embodiment, the patient is instructed to run a hot bath in a very clean bathtub before going to bed. The patient trunk and back portion of the head is to lay submerged in the hot water for approximately an hour. The temperature is such as to promote perspiration. The bath water is left undisturbed overnight to allow for conglomerates to form. The visible particles that form can be examined next morning for their fluorescent, electrostatic, magnetic and other ACE-pigment associated properties. As an adjunct to the hot bath method, the patient can be instructed to use a soap to generate lather on the skin and hair. A useful product is a commercial preparation of soap termed Miracle II neutralizer and soap (manufactured by Mr. Clayton Tedeton, Tedco Inc. L.A.). These particular products are advocated as cleansing agents. They have now been used in several patients as a means of promoting the elimination of ACE-pigments. An extension of the hot bath method is for the patient to subsequently sleep with clean nightwear on clean bed sheets. In the morning the patient is instructed to look for and to collect any particles that have adhered to the nightwear or bed sheets.

Urine isolations of ACE-pigments have also been performed on a few patients. Small but visible particles were seen developing in urine that was allowed to stand at room temperature for 48 hours. No bacterial growth was observed. If this were to be a problem, the urine could be passed through a Millipore filter. Similar processing could be applied to saliva, sputum or perspiration collected directly from the body.

More directly, a small amount of neutral red solution can be directly added to urine, saliva or plasma/serum specimens, followed by UV illumination. Alternatively, a fabric or Q-tip can be soaked with these bodily fluids and examined similarly to the method used for skin derived ACE-pigments. The determination is then made whether this addition of neutral red or other suitable dye evokes, or at least significantly enhances, the intensity of fluorescence occurring upon illumination with UV light.

A major use of the described methods for detecting the presence of reactive ACE pigments is to follow the course of a disease and to monitor the benefits of interventions aimed at enhancing the ACE pathway. Although more clinical studies are required, the presence of readily detectable reactive ACE pigments is viewed as an indication that this pathway can be beneficially activated in a patient. Conversely, therapy using, for example, UV light can be continued until no more fluorescing material is recoverable from areas where it was previously obtained.

All of the above methods will have applications to patients with a range of illnesses not only those that have an infectious component. The methods can also be applied to assess the status of the ACE pathway in animals.

The principles, preferred embodiments and modes of operation of the present invention have been described in the foregoing specification. The invention which is intended to be protected herein, however, is not to be construed as limited to the particular forms disclosed, since they are to be regarded as illustrative rather than restrictive. Additional advantages and modifications will readily occur to those skilled in the art. Variations and changes may be made without departing from the spirit of the invention encompassed by the appended claims.

What I claim as my invention is:

1. A method for assessing the status of the alternative cellular energy (ACE) pathway in a patient comprising the steps of i) collecting a sampling of ACE pigments from the skin and/or bodily fluids from the patient onto material, such as fabric, gauze or a Q-tip; ii) viewing the collected ACE pigments under ultraviolet (UL) light illumination, before and after exposure of the ACE pigments to a dye that selectively enhances the fluorescence of reactive ACE pigments, and with or without the use of a UV light transmitting filter that blocks visible light; and iii) interpreting the presence of fluorescing ACE pigments as an indication that the patient's ACE pathway is in a reactive status and that additional UV light stimulation and/or other methods are indicated to more fully energize the ACE pathway, while the absence of detectable fluorescence is an indication of the lack of current reactivity of the patient's ACE pathway to applied UL light illumination.

2. The method of claim 1 in which the collection of alternative cellular energy (ACE) pigments is made from the skin, with or without the additional step of promoting perspiration of the skin in the region from which the ACE pigments are being collected.

3. The method of claim 1 in which the collection of alternative cellular energy (ACE) pigments is made from saliva/sputum.

4. The method of claim 1 in which the collection of alternative cellular energy (ACE) pigments is made from urine.

5. The method of claim 1 in which the collection of alternative cellular energy (ACE) pigments is made from plasma/serum.

6. A method for collecting samples of alternative cellular energy (ACE) pigments from a subject in which such pigments are being produced and excreted in bodily fluids, including urine, saliva/sputum, plasma/serum and/or perspiration, comprising collection of the bodily fluid and allowing it to stand for a sufficient time for ACE pigment particles to form and then to subsequently collect such particles.

7. The method of claim 1 in which a compact spiral fluorescent light bulb is used as the source of ultraviolet light.

8. The method of claim 1 in which the ProLume compact spiral fluorescent light bulb is used as the source of ultraviolet light.

9. The method of claim 1 in which the fluorescence light is passed through a ultraviolet (UV) transmitting filter that blocks the transmission of any visible light component emitted from the ultraviolet light source, so as to create a dark background for the more sensitive detection of any UV induced fluorescence of reactive alternative cellular energy (ACE) pigments.

10. The method of claim 1 in which the UV transmitting filter is either Kodak Wratten 18A filter, Schneider Optics, New York, filter code number 403; or Rolyn Optics Company, Covina Calif., catalogue number 65.1030.

11. The method of claim 1 in which the alternative cellular energy (ACE) pigments are exposed to the dye neutral red prior to visualization for ultraviolet light induced fluorescence.

12. The method of claim 11 in which the alternative cellular energy (ACE) pigments are exposed to a freshly prepared neutral red solution ranging from 0.1 to 10 mg/ml prior to visualization for ultraviolet light induced fluorescence.

13. A method for collecting samples of UV reactive alternative cellular energy (ACE) pigments from a subject in which such pigments are being produced and excreted from the skin, comprising having the subject take a hot bath that promotes perspiration and collecting from the bath water pigmented materials released from the subject into the bath water, and subsequently having the subject sleep in clean nightwear and on clean bed sheets and collecting from the nightwear and bed sheets pigmented materials released from the subject's skin, including hair.
